(19) United States
(12) Patent Application Publication
(10) Pub. No.: US 2022/0115114 A1
(43) Pub. Date: Apr. 14, 2022

(54) Nutrition and Fitness System

(71) Applicant: Innes Imports Pty Ltd., Cowes (AU)

(72) Inventor: Marni Nicole Johnston, Cowes (AU)

(21) Appl. No.: 17/498,960

(22) Filed: Oct. 12, 2021

Related U.S. Application Data
(60) Provisional application No. 63/090,523, filed on Oct. 12, 2020.

(51) Int. Cl. G16H 20/60; G06Q 30/06

(52) U.S. Cl. CPC G16H 20/60; G06Q 30/0633; G06Q 30/0631

(57) ABSTRACT

A nutrition and fitness system has one or more computer devices and computer memory, the computer memory storing executable code that, when executed, enables the computer system to perform a process. The process includes the steps of first determining a basal metabolic rate of the user based upon his or her age, height, gender, and weight. Next, a plurality of food options and a plurality of fitness options are provided, and selected food options are received from the plurality of food options, and selected fitness options are received from the plurality of fitness options. A shopping list is provided of all of the ingredients necessary to prepare the selected food options selected by the user.

### CROSS-REFERENCE TO RELATED APPLICATIONS

This application for a utility patent claims the benefit of U.S. Provisional Application No. 63/090,523, filed Oct. 12, 2020.

### BACKGROUND OF THE INVENTION

### Field of the Invention

This invention relates generally to nutrition and fitness, and more particularly to a nutrition and fitness system that calculates and tracks a user's exercise and meals.

### Description of Related Art

Capps, U.S. 2021/0313066, teaches a health and fitness system for the generation and maintenance of a virtual assistant that facilitates a systematic and psychological approach to health-improvement and self-care. Via interaction with the assistant, patients interact to create personalized health-improvement plans and adapt based upon physiological measurements and artificial intelligence analyses of data collected from the patient via either a sensor or patient input. The system may dynamically improve health recommendations based upon patient response to interaction with the system. A computational method with statistical inference of biophysical parameters that define an artificial intelligence's internal biophysical simulation environment, which does not rely on an artificial neural network. The corresponding method relies on an internal biophysical network that computationally replicates the patient's unique, health-relevant physiological processes, and uses psychological techniques to encourage improvement of the patient's health.

### SUMMARY OF THE INVENTION

The present invention teaches certain benefits in construction and use which give rise to the objectives described below.

The present invention provides a nutrition and fitness system comprising one or more computer devices and computer memory, the computer memory storing executable code that, when executed, enables the computer device(s) to perform a process. The process includes the steps of first determining a basal metabolic rate of the user based upon his or her age, height, gender, and weight. Next, a plurality of food options and a plurality of fitness options are provided, and selected food options are received from the plurality of food options, and selected fitness options are received from the plurality of fitness options. A shopping list is provided of all of the ingredients necessary to prepare the selected food options selected by the user.

A primary objective of the present invention is to provide a nutrition and fitness system having advantages not taught by the prior art.

Another objective is to provide a nutrition and fitness system that plans meals and fitness activities, tracks the actual execution of these activities, and determines calorie surplus or deficit.

A further objective is to provide a nutrition and fitness system that utilizes the meal planning to provide a shopping list of food products needed for the preparation of the planned meals.

A further objective is to provide a nutrition and fitness system that allows for adjustment of the plan according to the actual activities of the user.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention.

FIG. 1 is a block diagram of a nutrition and fitness system according to one embodiment of the present invention.

FIG. 2 is a flow diagram illustrating the operation of the system.

FIG. 3 is a screen capture of an example of a meal planner generated by the system.

FIG. 4 is a screen capture of an example of a fitness planner generated by the system.

FIG. 5 is a screen capture of an example of a food tracker generated by the system.

FIG. 6 is a screen capture of an example of an exercise tracker generated by the system.

### DETAILED DESCRIPTION OF THE INVENTION

The above-described drawing figures illustrate the invention, a nutrition and fitness system that enables users to plan and track eating and exercise habits.

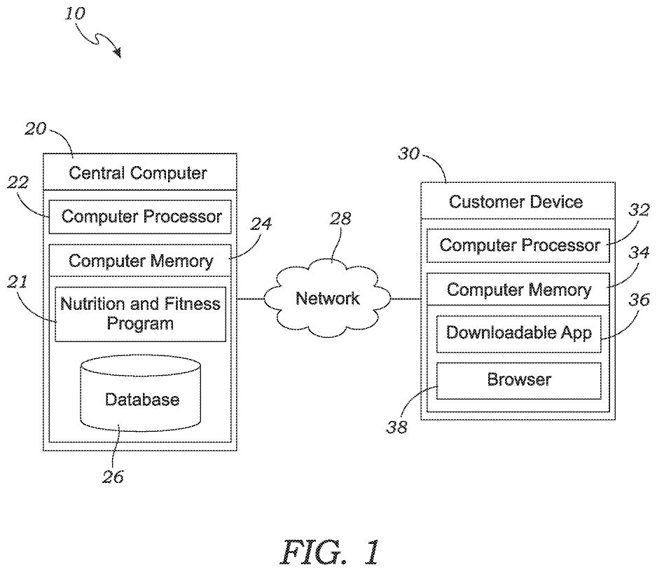

FIG. 1 is a block diagram of a nutrition and fitness system 10 according to one embodiment of the present invention. As shown in FIG. 1, the nutrition and fitness system 10 comprises a central computer 20 for enabling communications with a customer device 30 (e.g., smart phone, computer, laptop, tablet, or other similar device known in the art). The central computer 20 includes a computer processor 22 and a computer memory 24. The computer memory 24 includes executable code in the form of a nutrition and fitness program 21 that is discussed at length below. As illustrated, the computer memory 24 further includes or connects to a database 26 for storing data, also discussed in greater detail below.

As shown in FIG. 1, the customer device 30 includes a computer processor 32 and a computer memory 34, wherein the computer memory 34 may store a downloadable application 36 (a.k.a “app”) that communicates with the central server via a network 28 to run the nutrition and fitness program 21. The computer memory 34 may also include a browser 38 which may alternatively be used to interact with the nutrition and fitness program 21. In this embodiment, the central computer 20 and/or the customer device 30 may further be capable of communicating with a third party computer (not shown) via the network 28, discussed in greater detail below.

The terms “computer,” “computer device,” and similar terms used herein, refer to a device and/or system of devices that include at least one computer processing element, e.g., a central processing unit (CPU), and some form of computer memory having a capability to store data. The computer may comprise hardware, software, and firmware for receiving, storing, and/or processing data as described below. For example, a computer may comprise any of a wide range of digital electronic devices, including, but not limited to, a server, a desktop computer, a laptop, a smart phone, a tablet, or any form of electronic device capable of functioning as described herein.

The computer system may include one or more servers, desktop computers, multiprocessor systems, microprocessor-based or programmable electronics devices, network appliances, or any form of equivalent device(s) known in the art. The computer system may be in the form of a single device, or multiple devices. The central computer may be distributed over a plurality of network devices and/or implemented using cloud architecture. The system server may operate using a master/slave approach over a plurality of network devices, within a cluster, a peer-to-peer architecture, and/or any of a variety of other architectures.

The term “computer memory” as used herein refers to any tangible, non-transitory storage that participates in providing instructions to a processor for execution. Such a medium may take many forms, including but not limited to, non-volatile media, volatile media, and any equivalent media known in the art. Non-volatile media includes, for example, ROM, magnetic media, and optical storage media. Volatile media includes, for example, DRAM, which typically serves as main memory. Common forms of computer memory include, for example, hard drives and other forms of magnetic media, optical media such as CD-ROM disks, as well as various forms of RAM, ROM, PROM, EPROM, FLASH-EPROM, solid state media such as memory cards, and any other form of memory chip or cartridge, or any other medium from which a computer can read. While several examples are provided above, these examples are not meant to be limiting, but illustrative of several common examples, and any similar or equivalent devices or systems may be used that are known to those skilled in the art. Since the hardware for both the central computer 20 and customer device 30 is well known in the art, it is not described in greater detail herein, but may include any arrangement known in the art for purposes of this invention.

The term “database” as used herein, refers to any form of one or more (or combination of) relational databases, object-oriented databases, hierarchical databases, network databases, non-relational (e.g. NoSQL) databases, document store databases, in-memory databases, programs, tables, files, lists, or any form of programming structure or structures that function to store data as described herein.

The network 28 may include any device or system for communicating information from one computer device to another. For example, a global computer network (e.g., the Internet) may be used, including any form of local area networks (LANs), wide area networks (WANs), direct connections, such as through a universal serial bus (USB) port, other forms of computer-readable media, or any combination thereof. On an interconnected set of LANs, including those based on differing architectures and protocols, a router may act as a link between LANs, enabling messages to be sent from one to another. In addition, communication links within LANs typically include twisted wire pair or coaxial cable, while communication links between networks may utilize analog telephone lines, full or fractional dedicated digital lines including T1, T2, T3, and T4, Integrated Services Digital Networks (ISDNs), Digital Subscriber Lines (DSLs), wireless links including satellite links, or other communications links known to those skilled in the art. The network 28 may further include any form of wireless network, including cellular systems, WLAN, Wireless Router (WR) mesh, or the like. Access technologies such as 2G, 3G, 4G, 5G, and future access networks may enable wide area coverage for mobile devices. In essence, the wireless network 28 may include any wireless communication mechanism known in the art by which information may travel between computers of the present system.

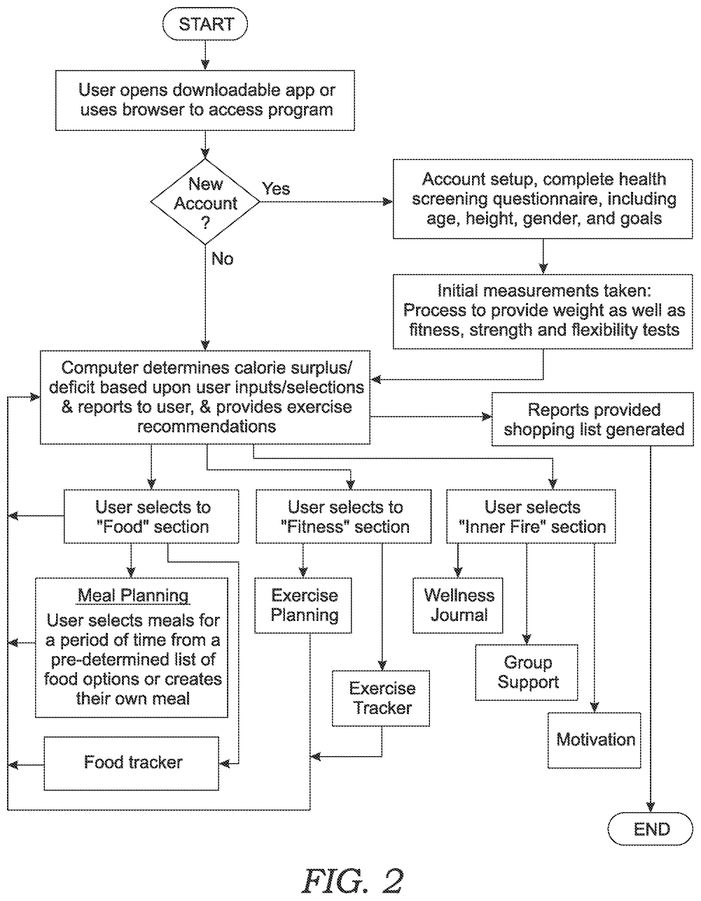

FIG. 2 is a flow diagram illustrating a process of the nutrition and fitness program 21. As is known in the art, the user may first download the downloadable app 36 of FIG. 1 from the central computer 20, or from an app store, or other computer mechanism known in the art. Alternatively, the browser 38 of FIG. 1 may be used on the customer device 30 to provide access to the nutrition and fitness program 21, or any other method of accessing the program 21 known in the art.

As shown in FIG. 1, in this embodiment, the application 36 prompts the user to either enter account information to gain access to an existing account, or to open a new account. The user is then navigated to choose a program option, sign up, pay, etc. There may be different program options having various features at a range of price points. For example, a user may sign up for just a nutrition element of the system 10, or just a fitness element, or a combination of certain features, or all features. Other features may be included in the options, e.g., community forums, video sharing, etc., or any other desirable user feature. The following description is for a scenario in which the user signs up for all features, but it should be understood that the user may use only certain features described herein.

To set up the account, the user may next be provided with a health questionnaire for adding information, such as the user's age, height, gender, and weight, then determining a basal metabolic rate of the user based upon his or her age, height, gender, and weight. The user may further enter other general information (e.g., name, contact information, preferences, etc.), at the accounts stage or after/within the questionnaire stage. The questionnaire of this embodiment gathers detailed information about if the user has a heart condition, high or low blood pressure diagnosis or symptoms, regular chest pains, or other chronic medical condition. The questionnaire of this embodiment further inquires about medications, musculoskeletal injuries, and doctor recommendations. Questions may also refer to respiratory issues, spinal cord injuries, stroke history, mental health problems/needs, metabolic conditions, cancer, arthritis, osteoporosis, etc., or any other details that may be relevant in a health screening. General medical history fields may be included to similarly enable the patient to add further information, particularly about important medical history, allergies, and similar factors. While some examples of data collection are discussed herein, the questionnaire may include several pages/sections gathering further information. The application 36 is obviously not limited to these examples, but should be construed to include equivalent forms of data, collected in any manner known in the art.

The central computer 20 shown in FIG. 1, or an equivalent computer device, then stores the account information and the answers from the questionnaires in the database 26 shown in FIG. 1. The user may also enter or select any fitness or nutrition goals they would like to reach. Example goals may be to lose or maintain current weight, reduce certain intakes, build muscle, increase flexibility, etc., or any other goal relating to nutrition and fitness.

As shown in FIG. 2, the user may be prompted to input current height and weight, and age and gender, to calculate his or her body mass index (BMI) and basal metabolic rate (BMR), to properly track progress and formulate a nutrition plan and a fitness plan. In some embodiments, user may be directed to take physical measurements on the spot to ensure accuracy. For example, a weigh-in process may be used, which may include a scale capable of transmitting data to the program 21, or another means. Other measurements may also be taken, e.g., waist or bicep circumference, etc., depending on the goals of the user.

In some embodiments, the nutrition and fitness program 21 provides a process for performing fitness, strength, and flexibility tests. This may be in the form of existing standardized fitness tests, or may be unique to the program 21 and/or adapted to the specific individual (i.e., based on age, weight, etc.). The tests may include exercises such as toe-touches, jumping jacks, etc., or any other exercises designed for the purpose of measuring a person's physical fitness.

At a next step of the program 21, the computer system determines an approximate goal calorie surplus/deficit based upon the information from the questionnaire, measurements, and fitness tests. The fitness data and the BMR may be added to the goals section to help calculate the number of calories the user may need. A “target” (number of calories) may be generated based on energy intake minus the energy deficit from exercise and normal daily energy loss. A report is generated for the user, wherein the user is able to select from fitness and nutrition recommendations. In this embodiment, the calorie surplus or deficit is reported by transmitting a digital report to the user. In alternative embodiments, the calorie surplus or deficit is instead or additionally reported by transmitting a digital report to a fitness consultant, so the consultant can review the user details and confirm whether the recommendations are appropriate. In such an embodiment, the consultant may have the ability to manually adjust or enter recommendations to the user.

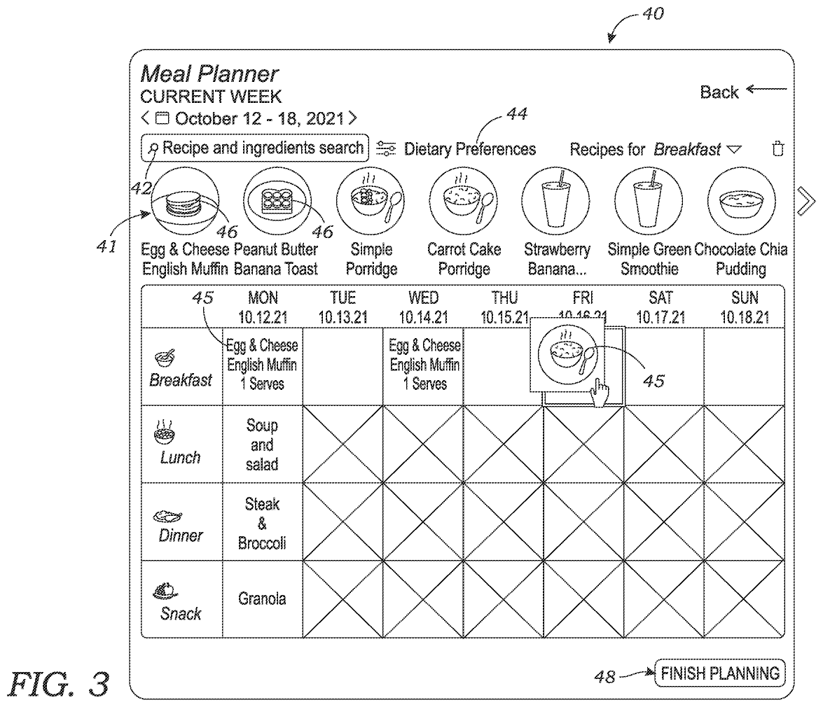

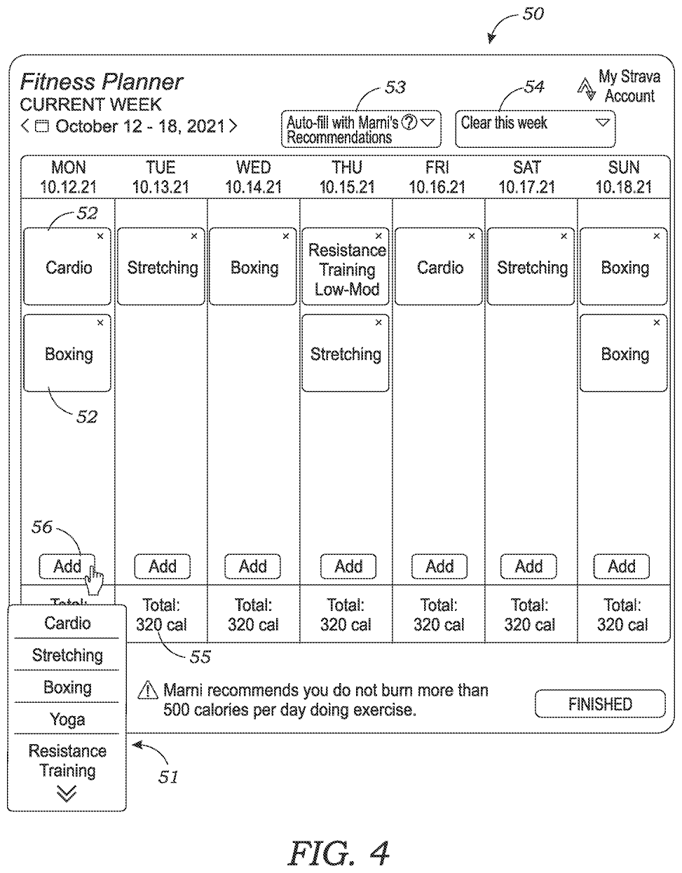

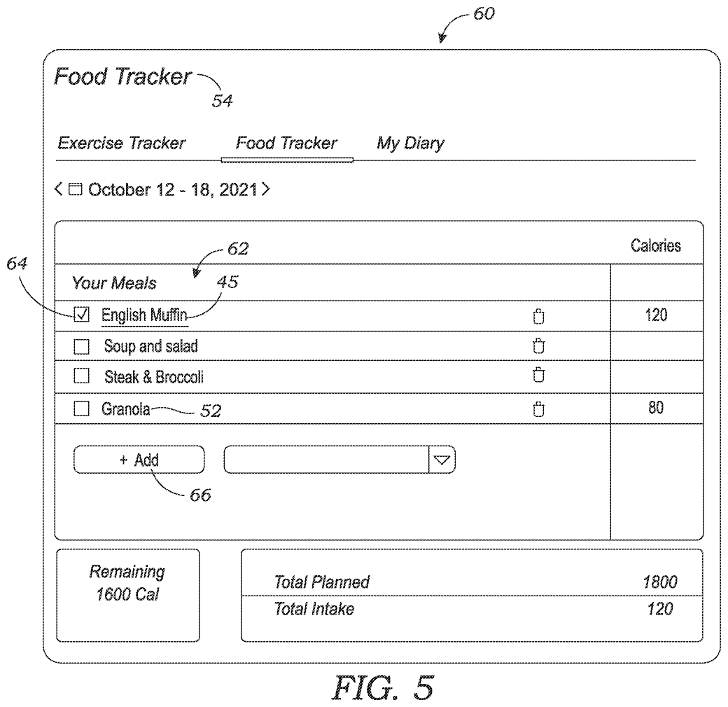

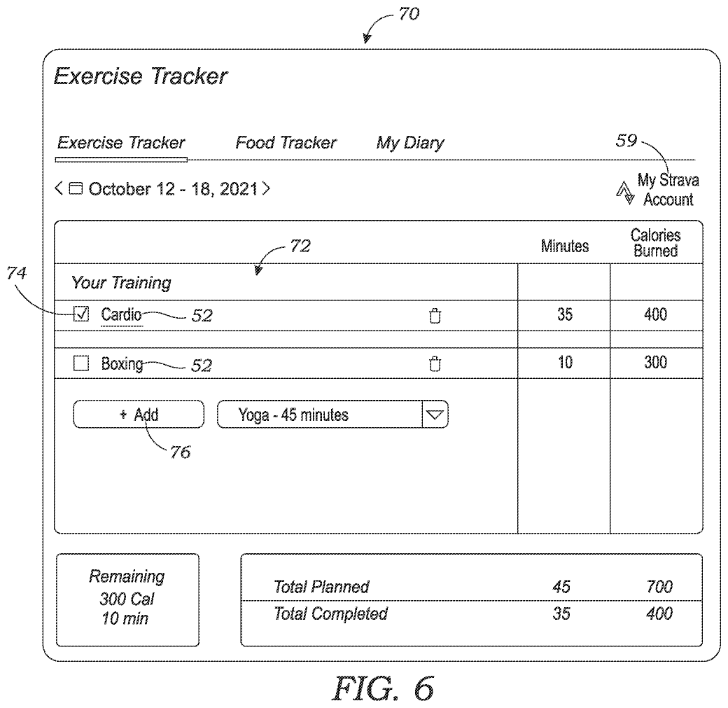

As shown in FIG. 2, once the report has been reviewed by the user and/or the consultant, the user selects or is directed to a “food” section, a “fitness” section, and/or an “inner fire” section. Each section may be completed in any order, and the steps described herein are merely an example of the process of using the program 21. In this embodiment, the food and fitness sections include a meal planner (shown in FIG. 3), a fitness tracker (shown in FIG. 4), as well as a food tracker (shown in FIG. 5), and an exercise tracker (shown in FIG. 6).

“Inner fire” refers to psychological health and support, and may include features such as a wellness journal (i.e., digital log), and a feature that enables interaction with a community of users to share tips, difficulties, or anything else compatible with the group support format. In some implementations of the present invention, the inner fire section also includes a motivation element, which may be in the form of videos or images of encouragement or inspiration, and may further involve the participation of multiple users or consultants. Obviously, the term “inner fire” may be replaced by similar terms such as “mental wellness,” “personal support,” etc., or any other similar terms intended to describe the related features.

In various embodiments, other sections/pages may be included, e.g., other forms of progress tracking, bonus content, account info, customer support, guides, etc., or any other features commonly associated with this type of website.

FIG. 3 illustrates an example embodiment of a meal planner 40 generated by the system 10 of FIG. 1. As shown in FIG. 3, the meal planner 40 displays a plurality of food options 41, which may include meal options and/or recommendations based on the set goals. The central computer 20 (of FIG. 1) may reference the database 26 for caloric information on meal options, information on macro/micro nutrients, etc., and any other information relevant to nutrition.

As shown in FIG. 3, recipes/meals and ingredients and their respective caloric and nutritional data may be searchable by the user using a search bar 42. The user may follow the recommendations to hit precise caloric and nutritional targets, or the user may alternatively use the recommendations as a general guideline, and add whatever is desired to the meal planner 40.

As shown in FIG. 3, the user may have an option 44 to select dietary preferences (vegetarian, gluten-free, allergies, flavor/texture preferences, etc.) so that the program 21 may provide the appropriate food options. Other preferences may also be input, according to the needs of the consumer. The user may also choose which meals (breakfast, lunch, snacks, and/or dinner) will be planned. A further option (not shown) may be provided to select the number of persons each meal will serve, so a user may order for an additional person or group of people.

Recommended options 46 may be provided in visual form, text, or any other manner known in the art.

The meal plan may be for a full week, or other set amount of time (e.g., daily, bi-weekly, monthly, etc.). Selected food options 45 may be inserted (e.g., drag and drop, etc.) into the calendar format, so that the user may select each date to plan a meal(s) for that date. However, other formats may also be provided for meal selection, i.e., in a list, table, etc. During meal selection, the central computer 20 may reference the database 26 for caloric information so the user may select items that are compatible with the target. In use, unplanned food may be added by searching the database 26 or by manually entering the ingredients.

In some embodiments, the user may manually add recipes or ingredients wherein an option (not shown) to share the recipe or ingredient data may further be included. If the user wishes to share a recipe, they may select this option wherein the recipe will be sent to a consultant to verify that it is suitable for the broader community to access.

After the user has selected desired meal options for the week, he or she may select a “finish planning” option 48, or similar option such as “complete,” “proceed,” “done,” etc.

The user may elect to generate a shopping list at that time, or later, which facilitates gathering ingredients for the meals selected. This may be done manually with a printed list. However, in some embodiments, the system 10 interacts with a third party computer via the network 28, wherein appropriate access permissions and/or sign-in may be required. The third party computer may be associated with an online grocery service that offers pickup or delivery of selected items, which may be from any compatible grocery store. Once the shopping list is generated, the user may adjust the selections, and may also add items not related to the meal plan, so that general shopping (i.e., for household supplies, or any other purchases) may be completed at the same time as ordering ingredients for the planned meals.

The grocery service may be integrated with the system 10 such that it may be accessed directly through the program 21, or the app/browser 36/38 may redirect the user to another merchant site to complete the order. The user arranges payment of the items and selects his or her preference for pickup or delivery. Such ordering services are well known in the art, and any commonly-found features should be considered compatible with the present invention. Most existing mealkit services have their own supply chains for food delivery and as such price their food and memberships well above the retails price of comparable ingredients from retail grocery stores. The nutrition and fitness program 21 is capable of connecting to a standard retail online ordering service. Other online ordering services may be included outside of groceries, as many fields may benefit from this particular implementation of a virtual shopping system, e.g., yoga equipment, vitamins, etc. Further third party services may be integrated, e.g., a music streaming service, or other fitness support services (e.g., FITBIT, etc.), discussed below.

After receiving the groceries, the user may prepare meals based on the plan and/or recipes provided by the program 21 until it is time to make another order, wherein the user may repeat the same order as before or create a new plan. In some embodiments, the user may review past meal plans and select a previous order, or generate a list with new items. In some embodiments, the user can manage favorite meal plans by “liking” a past meal plan or individual meal (not shown).

FIG. 4 is a screen capture of an example of a fitness planner 50 generated by the system 10. As shown in FIG. 4, in this embodiment, the user may plan physical exercises 52 according to the targets generated by the program 21 in the previous steps. The fitness planner 50 may be similar to the meal planner 40, but may be shown in various different format as described above.

As shown in FIG. 4, in this embodiment, the user may receive a plurality of exercise options 51, and select selected exercise options 52 for each day, according to the set goals and target calories. The selected exercise options 52 may be manually selected by the user, or provided by the system. For example, recommended options may be auto selected via button 53, or selected options may be cleared with a clear button 54. A button 56 may be used to add further options manually. The library and/or schedule of exercise classes may include different exercise programs like endurance, strength, stretching, etc., or any other type of exercise activity that may be logged. Similar to the discussion of FIG. 3, the user may save exercises or week plans to use again later. In some implementations, the user can also submit exercises to be reviewed and potentially shared with the community (once approved). However, the user may also have the option of keeping submitted exercises private.

FIG. 5 is a screen capture of an example of a food tracker 60 generated by the system. As shown in FIG. 5, a list of selected foods 62 is provided where the selected foods that were selected in the meal planner are auto-populated into this tracker 60. As shown in FIG. 5, the food tracker 58 may include a check box 64 or equivalent mechanism that enables the user to “tick” off (or otherwise mark) pre-planned meals/ingredients from the food tracker 58 as they are consumed. An add button 66 may be included to manually add foods that the user consumes. The database 26 may automatically calculate calories and/or other nutritional information, or these may also be entered manually. Total calories consumed, as well as calories planned, and calories remaining may be included in the food tracker 58 for reference.

FIG. 6 is a screen capture of an example of an exercise tracker 70 generated by the system. As shown in FIG. 6, a list of selected exercises 72 is provided where the selected exercises that were selected in the meal planner are auto-populated into this tracker 70. The exercise tracker 70 may be used to log exercises performed, via a check box 74 or equivalent mechanism, and an add button 76 may similarly be used to manually add exercises that are performed outside of the plan.

If the user chooses to watch a video or otherwise follow instructions provided by the system 10, the duration in minutes and number of calories burned may be auto-filled. However, the user may also add exercises, including number of minutes, manually. In this embodiment, total calories burned and total calories remaining may be listed so the user can monitor whether he or she is hitting the target throughout the day.

In some embodiments, third-party applications may be connected to the nutrition and fitness program 21. For example, software that enables a wearable device to track exercise and caloric expenditure may be connected such as STRAVA (or similar apps., or music programs such as SPOTIFY, ITUNES, etc.) may be connected so the user can play music while performing the exercises. App. permissions or a separate account may be required to connect the program 21 to a third-party application.

In use, the user follows the provided plans and periodically inputs calories consumed, exercises completed, weigh-ins, etc., and the central computer 20 records progress data in the database 26 and provides feedback to the user, e.g., graphically, serially, etc. The user may review the feedback provided by the program 21 to determine if they wish to continue the existing nutrition and fitness plans, or if they would like to adjust the plans. The program 21 may further provide said data in a graph format, so the user can see changes in weight, BMI, girth, etc. In some embodiments, the user may perform fitness tests, the results of which may also be provided in a graph or other format. In this manner, the user is able to see, in real time, the effectiveness of the provided nutrition and fitness plans.

Using new feedback from the user (e.g., set preferences, remove or add meal/workout/etc.), the central computer 20 and/or a consultant may generate a new nutrition and/or fitness plan and transmit it via the network 28 to the customer device 30. The user may repeat this step until satisfied with the provided plans, wherein he or she may continue indefinitely or until set goals are reached.

It should be understood that the web pages and/or app screens illustrated herein are merely exemplary embodiments, and those skilled in the art may devise many alternative web pages, or include additional options (e.g., settings, profile, history, etc.) in a sidebar or in an alternative screen, as is well-known in the art. While certain embodiments of web pages are illustrated, the system 10 should not be limited to this disclosure, but should be construed to include alternative embodiments which may be designed by one skilled in the art.

The disclosed embodiments may be embodied in many different forms and should not be construed as limited to the embodiments set forth herein; rather, these embodiments are provided so that this disclosure will be thorough and complete, and will fully convey the scope of the embodiments to those skilled in the art. Among other things, the various embodiments may be methods, systems, media, devices, or any similar or equivalent arrangements known to those skilled in the art. Accordingly, the various embodiments may take the form of an entirely hardware embodiment, an entirely software embodiment or an embodiment combining software and hardware aspects. The following detailed description is, therefore, not to be taken in a limiting sense.

The title of the present application, and the claims presented, do not limit what may be claimed in the future, based upon and supported by the present application. Furthermore, any features shown in any of the drawings may be combined with any features from any other drawings to form an invention which may be claimed.

As used in this application, the words “a,” “an,” and “one” are defined to include one or more of the referenced item unless specifically stated otherwise. The terms “approximately” and “about” are defined to mean +/−10%, unless otherwise stated. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by claims made to the invention.

What is claimed is:

1. A nutrition and fitness system comprising one or more computer devices and computer memory, the computer memory storing executable code that, when executed, enables the computer system to perform a process that comprises:
receiving a user's age, height, gender, and weight;
determining a basal metabolic rate of the user based upon his or her age, height, gender, and weight;
providing a plurality of food options and a plurality of fitness options;
receiving selected food options from the plurality of food options;
receiving selected exercise options from the plurality of fitness options;
generating a shopping list of all of the ingredients necessary to prepare the selected food options selected by the user; and
providing the shopping list.

2. The nutrition and fitness system of claim 1, wherein the shopping list is provided in the form of a digital list of food ingredients that is transmitted to the user.

3. The nutrition and fitness system of claim 1, wherein the shopping list is provided by transmitting an order to a grocery store for delivery to the user.

4. The nutrition and fitness system of claim 1, wherein the executable code performs the additional steps of:
determining calorie surplus or deficit based upon the selected food options, the selected fitness options, and the user's basal metabolic rate; and
reporting the calorie surplus or deficit.

5. The nutrition and fitness system of claim 4, wherein the calorie surplus or deficit is reported by transmitting a digital report to the user.

6. The nutrition and fitness system of claim 4, wherein the calorie surplus or deficit is reported by transmitting a digital report to a fitness consultant.

7. The nutrition and fitness system of claim 1, wherein the executable code performs the additional steps of:
providing an exercise tracker to track when the selected fitness options are performed by the user;
providing a food tracker to track when the selected food options are consumed by the user;
receiving data from the exercise tracker and the food tracker to track calorie intake and expenditure;
determining actual calorie surplus or deficit based upon user's actual activities; and
reporting the actual calorie surplus or deficit.
